(19) United States
(12) Patent Application Publication
(10) Pub. No.: US 2019/0117651 A1
(43) Pub. Date: Apr. 25, 2019

(54) COMPOSITION FOR STIMULATING SIRTUIN ACTIVITY

(71) Applicant: Bitop AG, Witten (DE)

(72) Inventors: Andreas BILSTEIN, Bergheim (DE); Jean KRUTMANN, Düsseldorf (DE); Klaus UNFRIED, Düsseldorf (DE)

(73) Assignee: Bitop AG, Witten (DE)

(21) Appl. No.: 16/083,909

(22) PCT Filed: Mar. 13, 2017
(86) PCT No.: PCT/EP2017/055843

§ 371 Date: Dec. 13, 2018

(30) Foreign Application Priority Data
Mar. 11, 2016 (DE) 10 2016 104 470.9

(51) Int. Cl. A61K 31/505; A61K 9/00; A61K 9/127; A61K 9/51

(52) U.S. Cl. CPC A61K 31/505; A61K 9/51; A61K 9/127; A61K 9/0073

(57) ABSTRACT

The invention relates to a composition containing as active agent ectoine, hydroxyectoine and/or salts, esters or amides of these compounds for the stimulation of sirtuin activity, in particular for the treatment of diseases that are associated with reduced activity of sirtuins. These include in particular various aging phenomena and diseases that occur primarily in old age or are associated with an impaired cell differentiation or cell proliferation.

The invention relates to a composition for the stimulation of the activity of sirtuins, in particular for the treatment and/or prophylaxis of diseases associated with reduced activity of sirtuins. These are in particular signs of aging as well as diseases linked with impaired cell differentiation.

Sirtuins are mostly histone deacetylases of type III, which occur in almost all life forms in highly conserved evolutionary form. The name derives from the term “silent information regulator”. Sirtuins deacetylate lysine residues, in particular histone proteins at the N-terminal end, which, as a component of chromatin, are responsible for packaging the DNA, where NAD+ is required as a cofactor for deacetylation. Therefore, aside from the deacetylated protein nicotinamide and O-acetyl-ADP-ribose develop after elimination of the acetyl group. Because the lysine carries a positive charge after deacetylation, the affinity of the histone to the negatively charged backbone of the DNA increases. The DNA is thus more strongly blocked resulting in the transcription being reduced. Sirtuins therefore have a direct influence on cellular processes as they are able to regulate the expression of certain proteins and enzymes. A total of 7 different sirtuins, i.e. SIRT1 to SIRT7, are distinguished in humans and other mammals. In addition to histones, intracellular signal molecules of sirtuins, for example, are deacetylated as well.

Due to the influence of sirtuins on the expression of proteins and enzymes, which are partly associated with certain diseases, a great deal of research efforts has been focused on this enzyme class for several years. In particular, sirtuins appear to have a positive effect on various aging phenomena or diseases that more frequently occur in old age, for example because they are associated with altered gene regulation in cell differentiation processes. For this reason, the press has already spoken of “anti-aging enzymes” in this context and, among other things, the effects of sirtuin activators, such as resveratrol, are being investigated.

The diseases over which sirtuins have an influence include the already mentioned aging phenomena, particularly those of the lungs such as the occurrence of senile emphysema, which is also called age emphysema or primary atrophic emphysema. Aside from the aging of the lung, it has also been discussed in literature the connection between sirtuins and other diseases, such as heart hypertrophy, gout, atherosclerosis or arteriosclerosis, obesity, amyotrophic lateral sclerosis or cancer, especially in the field of hematology such as acute or chronic leukemia or malignant lymphomas. The regulation of gene activity via histones plays an important role in cell differentiation. A connection was postulated between an aging or disease-related loss of sirtuin activity and the occurrence of aging symptoms.

It is therefore the objective of the invention to provide substances or compositions that counteract the age-related loss of sirtuin activity.

Surprisingly, it has been discovered that ectoine, hydroxyectoine and salts, esters and amides of ectoine/hydroxyectoine are capable of significantly reducing the loss of sirtuin activity. Primarily, sirtuin activity refers to the amount, structure and/or function of sirtuins, i.e. reduced sirtuin activity may be encountered if sirtuins are less active but also if fewer sirtuins are present. In this way, both therapeutic treatment and a preventive effect can be brought about.

Ectoine and hydroxyectoine are tetrahydropyrimidine derivatives which are synthetized under stress conditions in extremophilic, especially halophilic microorganisms. Various applications or uses have been described hitherto for ectoine and hydroxyectoine, for example as moisturizers, for the treatment of the vascular leak syndrome (VLS) (DE 10 2006 056 766 A1) or for the treatment of neurodermatitis (DE 103 30 243 A1). The systematic name of ectoine is 2-methyl-1,4,5,6-tetrahydropyrimidine-4-carboxylic acid and of hydroxyectoine 5-hydroxy-2-methyl-1,4,5,6-tetrahydropyrimidine-4-carboxylic acid.

The structure of natural L-ectoine ((S)-2-methyl-1,4,5,6-tetrahydropyrimidine-4-carboxylic acid) is illustrated below:

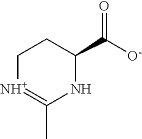

The structure of natural hydroxyectoine ((4S,5S)-5-hydroxy-2-methyl-1,4,5,6-tetrahydropyrimidine-4-carboxylic acid) is indicated hereunder:

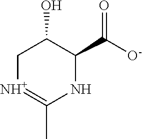

The use of the stereoisomers indicated is preferred but not obligatory, i.e. other stereoisomers or racemates may also be employed.

The composition proposed by the invention can generally be adopted for the treatment and/or prophylaxis of aging symptoms and for influencing self-renewal processes, cell differentiation and cell proliferation. The composition is of particular importance in the treatment of aging symptoms of the lung, especially of the senile emphysema. This is characterized by the degeneration of the alveolar structures in the lungs, which leads to a reduction of the lung surface which is available for the exchange of gas. In such cases, the patient finds it hard to breathe properly. It could be shown that the repeated application of a solution containing ectoine into the lungs leads to a significant reduction of the loss of sirtuin activity in the lung tissue in rats. Although the treatment of lung diseases caused by the action and influence of suspended dust has already been described in publication WO 2005/002556 A1, it could surprisingly be ascertained that also general lung aging symptoms can be treated with ectoine/hydroxyectoine. The aging of tissue is attributable to an altered gene activity during the self-renewal and cell differentiation processes in (lung) tissue, which is regulated by the accessibility of DNA by histones and is thus also dependent on sirtuins.

Accordingly, the treatment/prophylaxis of pulmonary diseases resulting from exposure to suspended particulate matter may be excluded from the present invention, as can be learned from what has been disclosed in WO 2005/002556 A1. Likewise excluded may be the treatment/prophylaxis of pneumonia, asthma, COPD, ARDS (Acute Respiratory Distress Syndrome), cystic fibrosis, pulmonary fibrosis, silicosis, sarcoidosis, allergies and bronchial hyperreactivity, as can be seen from WO 2013/034299 A1.

Another group of lung diseases that can be treated with or prevented by the composition proposed by the invention is the group of idiopathic interstitial pneumonias (IIPs). The cause of these diseases is not known, which is why they are called idiopathic. They mainly affect the connective tissue of the lungs. Patients suffer from respiratory distress, which in the initial stage can be limited to physical strain, but which often worsens over time and turns into respiratory insufficiency in the final stage. As a result, there may be a lack of oxygen in the blood. These diseases are also frequently associated with an impaired cell proliferation.

According to the current classification, a distinction is made between seven different forms of the disease:
Idiopathic pulmonary fibrosis (IPF)
Non-specific interstitial pneumonia (NSIP)
Cryptogenic organizing pneumonia (COP)\
Acute interstitial pneumonia (AIP)
Respiratory bronchiolitis with interstitial lung disease (RB-ILD)
Desquamative interstitial pneumonia(DIP) and
Lymphoid interstitial pneumonia (LIP).

In addition, there are unclassifiable IIPs for diseases that cannot be clearly defined.

Idiopathic pulmonary fibrosis (IPF) is particularly widespread. The treatment using the composition in accordance with the invention also plays a special role here because the disease can hardly be overcome with the aid of anti-inflammatory drugs. The same applies to acute interstitial pneumonia (AIP). Normally, both diseases have a poor prognosis, which is why the treatment of these diseases according to the invention is of special significance.

Among the aging phenomena which can be treated or prevented with the help of the composition offered by the invention are also intrinsic skin aging phenomena. These are to be understood as signs of skin aging that are not the result of external influences such as UV radiation or general solar radiation. Rather, they are caused by a reduced reactivity of the skin cells, are genetically determined and usually begin at the age of 30 at the latest. The skin loses elasticity and resilience. Due to the reduced cell proliferation of the skin at an advanced age, the content of collagen and elastin in the skin decreases considerably.

Moreover, skin aging caused by airborne suspended particulate matter can also be treated or prevented by the composition according to the invention. In particular suspended particulate matter (also called fine dust) with an average particle size of ≤15 μm, especially ≤10 μm may cause skin aging symptoms. Such suspended particulates are often produced by the combustion of fossil fuels, but also occur in the form of sand, spores, pollen, rock dust, in agriculture, mining, through tobacco consumption, tire abrasion, brake abrasion or forest fires.

Generally speaking, the invention covers in particular the treatment/prophylaxis of aging symptoms and diseases ascribed to impaired cell differentiation or cell proliferation, which are influenced or caused intracellularly by endogenous factors. The strengthening of the expression of sirtuins by ectoine/hydroxyectoine improves the cell differentiation of the tissue.

All human epithelia and endothelia that come into contact with ectoine/hydroxyectoine are positively influenced in their self-renewal and cell differentiation. Accordingly, the invention concerns a composition for the treatment of endothelial, epithelial and neurological diseases. Aside from the diseases already mentioned hereinbefore, other diseases exist that can be described as signs of aging in the sense that they primarily or rather often occur in humans at an advanced age, or that are associated with impaired cell differentiation/cell proliferation. These include: hypertrophy of the heart, gout, arteriosclerosis/atherosclerosis, obesity, amyotrophic lateral sclerosis (ALS) and cancer. Cancers in this context are diseases mainly occurring in the field of hematology such as acute or chronic leukemia and malignant lymphomas. Epithelial and mesenchymal tumors, i.e. carcinomas and sarcomas in general, can also be treated. Moreover, as treatable cancers are also to be mentioned: Lung cancer, skin cancer, intestinal cancer (especially colon cancer), breast cancer, prostate cancer, pancreatic cancer, esophageal cancer, laryngeal cancer, gastric cancer, cervical cancer, ovarian cancer, testicular cancer, kidney cancer, bladder cancer, thyroid cancer, oral cavity cancer, pharyngeal cancer and brain tumors.

Further treatable diseases in the field of hematology are anemia or MDS (myelodysplastic syndrome). Ectoine/hydroxyectoine is also suitable for the treatment and prophylaxis of these diseases. The therapy of amyotrophic lateral sclerosis is based on the fact that sirtuins show a stimulating effect on the growth of neurons and axons.

The sirtuin, the activity of which is increased or whose activity loss is reduced, is in particular SIRT-1, which has cardioprotective, cancer-inhibiting, anti-inflammatory and antioxidative effects. Aside from this, SIRT6 is of particular importance. It could be demonstrated that a deficiency of these sirtuins leads to premature aging in mice (Cheng et al., Proc Natl. Acad Sci USA 2003, 100, 10794-10799; Mostoslaysky et al., Cell 2006, 124, 315-329).

Pharmacologically compatible salts of the ectoine/hydroxyectoine embrace alkaline or alkaline-earth salts, in particular the salts of potassium, sodium, magnesium and calcium but also salts with organic bases such as, for example, with non-toxic aliphatic or aromatic amines.

Through the reaction of the carboxyl group of the ectoine/hydroxyectoine with alcohols or amines relevant esters or amides can be obtained which may also be employed within the scope of the invention. In this case, the COOH group of ectoine/hydroxyectoine is replaced by a carboxylic acid ester function COOR or a carboxylic acid amide function CONHR' or CONR′R″, where R, R′, R″ independently of one another represent saturated or unsaturated, straight-chain or branched alkyl, cycloalkyl, aryl, heteroaryl, alkylaryl, arylalkyl, alkoxyalkyl, alkylthioalkyl, aryloxyalkyl or arylthioalkyl groups. In particular, it may be a C₁, C₂, C₃, C₄, C₅, C₆, C₇, C₈, C₉, C₁₀, C₁₁, C₁₂, C₁₃, C₁₄, C₁₅, C₁₆, C₁₇ or C₁₈ alkyl group. In case of hydroxyectoine also the hydroxy group may be subjected to a reaction with a carboxylic acid of varying chain length to form a relevant ester. Also, the respective esters or amides may be present in ionic or zwitterionic form, i.e. the invention includes the use of salts of the said esters or amides.

The ectoine amide of 2-hydroxy-5-aminobenzoic acid may be used as well. The structural formula is as follows:

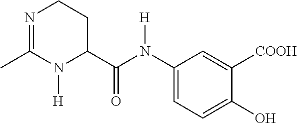 [SMILES: CC1=NCCC(C(=O)Nc2ccc(O)c(C(=O)O)c2)N1]

Therefore, this is the 2-methyl-1,4,5,6-tetrahydropyrimidine-4-carboxylic acid amide of the 2-hydroxy-5-aminobenzoic acid. Preferably, it is the relevant amide of the L-ectoine: (S)-2-methyl-1,4,5,6-tetrahydropyrimidine-4-carboxylic acid amide. The use of hydroxyectoine amide of the 2-hydroxy-5-aminobenzoic acid is also conceivable.

The composition may contain customary auxiliary substances, for instance carrier agents, preservation agents, bactericides, solutizers, vitamins, stabilizers, anti-foaming substances, osmotically active substances, colorants, surfactants, emulsifiers, moisturizing substances, viscosity increasing agents or the like. In the event of an administration in solid form, for example by means of powder inhalers it is recommendable that only carrier substances are used that are easily resorbed and non-irritating such as micronized lactose.

For example, preservation agents include thiomersal, organic mercury compounds such as phenylmercury, benzalkonium chloride, chlorhexidine, benzyl alcohol, glucose, ethanol and quaternary ammonium salts.

The formulations within the scope of the invention may also contain suitable buffer systems or other auxiliary substances for pH adjustment in order to reach and maintain a desired pH value. Typically, the pH values range between 4 and 8, preferably between 5 and 7.5, more preferred are approx. 7. Suitable buffer systems are citrate, phosphate, TRIS, glycine, borate, acetate. These buffer systems may be produced from substances such as citric acid, monosodium phosphate, disodium phosphate, glycine, boric acid, sodium tetraborate, acetic acid or sodium acetate.

While the compositions may contain further active agents it is, however, easily possible and sufficient for the treatment or prevention of the aging symptoms to use compositions that only contain as active substances ectoine, and/or hydroxyectoine, respectively salts, esters or amides thereof.

The concentration of ectoine/hydroxyectoine and/or corresponding salts, esters or amides can, in particular, be in a range of between 10 and 500 mM, preferably between 50 and 500 mM, especially preferred between 100 and 500 mM or between 100 and 200 mM. These concentrations have proven to be suitable for achieving the inventive effect. In particular, the proportion of ectoine/hydroxyectoine and/or corresponding salts, esters or amides in the composition may be in a range from 0.05 to 20% w/w, preferably from 0.1 to 10% w/w. A good effect could be observed, for example, in a range between 0.5 and 2% w/w.

The composition may be provided in the form of a solution, preferably an aqueous solution. Also possible is the presence as suspension, emulsion or microemulsion.

In order to improve the application and the shelf life of the composition provided in accordance with the invention, the composition containing the active ingredient can also be encapsulated in nanostructures or administered in the form of liposomes. This is particularly advantageous if the composition does not contain any preservative agents. Suitable methods for the encapsulation and production of liposomes are generally known from the state of the art.

Furthermore, the invention also concerns a spray device by means of which the composition provided by the invention can be inhaled. The spray device is provided with means for atomizing the composition and has an outlet opening that allows the user to inhale the composition. A spray device is particularly important in connection with the treatment or prevention of aging symptoms of the lungs, especially the senile emphysema. Suitable spraying devices can serve and be used for inhalation of an aerosol or powder. Powder inhalers include for example, the Spinhaler, Diskhaler, Turbohaler, Rotahaler or aerolizers.

1. Composition containing as active agent ectoine, hydroxyectoine and/or salts, esters or amides of these compounds for stimulating the activity of sirtuins.

2. Composition containing as active agent ectoine, hydroxyectoine and/or salts, esters or amides of these compounds for use in a method of treatment and/or prophylaxis of diseases that are associated with reduced activity of sirtuins.

3. Composition according to claim 1-, characterized in that the sirtuin is SIRT-1 or SIRT-6.

4. Composition containing as active agent ectoine, hydroxyectoine and/or salts, esters or amides of these compounds for use in a method for the treatment and/or prophylaxis of aging symptoms and/or diseases that are ascribable to impaired cell differentiation or cell proliferation.

5. Composition for use according to claim 4, characterized in that the aging symptoms are signs of aging phenomena of the lungs.

6. Composition for use according to claim 5, characterized in that the aging phenomenon of the lung is a senile emphysema.

7. Composition for use according to claim 4, characterized in that the aging phenomena are aging symptoms of the skin that are ascribable to the influence of suspended particulate matter or intrinsic aging phenomena of the skin.

8. Composition for use according to claim 4, characterized in that the symptoms of aging or the diseases that are ascribable to impaired cell differentiation or cell proliferation are cardiac hypertrophy, gout, arteriosclerosis or atherosclerosis, obesity, amyotrophic lateral sclerosis, cancer or hematological diseases.

9. Composition for use according to claim 8, characterized in that the cancers or hematological diseases are selected from: acute or chronic leukemia, malignant lymphoma, anemia and MDS (myelodysplastic syndrome).

10. Composition containing as active agent ectoine, hydroxyectoine and/or salts, esters or amides of these compounds for use in a method for the treatment and/or prophylaxis of an idiopathic interstitial pneumonia (IIP).

11. Composition for use according to claim 10, characterized in that the idiopathic interstitial pneumonia is idiopathic pulmonary fibrosis (IPF) or acute interstitial pneumonia (AIP).

12. Composition for use according to claim 1 for an application through inhalation.

13. Composition for use according to claim 1, characterized in that the concentration of ectoine, hydroxyectoine and/or salts, esters or amides of these compounds in the composition ranges between 10 and 500 mM, preferably between 100 and 500 mM.

14. Composition for use according to claim 1, characterized in that the composition is encapsulated in nanostructures or is provided in the form of liposomes.

15. Spray device containing a composition for use according to claim 1 for application of the composition over a patient's respiratory tract.

16. Composition according to claim 2, characterized in that the sirtuin is SIRT-1 or SIRT-6.
